(19) United States
(12) Patent Application Publication
(10) Pub. No.: US 2018/0008208 A1
(43) Pub. Date: Jan. 11, 2018

(54) Patient-Assisted Alarm System

(71) Applicant: General Electric Company, Schenectady, NY (US)

(72) Inventors: Mika Sarkela, Helsinki (FI); Rene Johannes Coffeng, Helsinki (FI); Kimmo Uutela, Helsinki (FI); Liisa-Maria Järvelä, Helsinki (FI)

(21) Appl. No.: 15/205,539

(22) Filed: Jul. 8, 2016

(51) Int. Cl. A61B 5/00; A61B 5/026; A61B 5/0215

(52) U.S. Cl. CPC A61B 5/746; A61B 5/747; A61B 5/742; A61B 5/0215; A61B 5/6843; A61B 5/026; A61B 5/7282

(57) ABSTRACT

In the present invention, a system and associated method is provided for monitoring vital parameters of a patient. The monitoring system includes sensors disposed on the patient and operably connected to a monitor. The parameters that are sensed by the sensors are transmitted to the monitor and compared with alarm thresholds and operational criteria stored within the monitor. When an alarm condition is sensed by the system, the system can actively solicit patient assistance in the confirmation of the alarm condition based on a set of reactive inputs stored within the system to enable medical personnel to appropriately respond to clinically relevant sensed alarm condition(s).

### BACKGROUND OF INVENTION

The invention relates generally to monitoring devices and equipment for obtaining and illustrating data about a patient to which the equipment is connected, and more particularly to monitoring devices and methods for obtaining patient assistance in ascertaining whether a triggered alarm is a clinically relevant alarm.

In monitoring or diagnostic devices that are currently utilized, the data obtained by the devices is compared to a set limit or threshold for the particular physiologic parameter being measured and represented by the incoming data to the device. When the data exceeds the limit the device is configured to trigger or set off an alarm in order to indicate the current condition of the patient to a treating physician or other medical care professional that is monitoring the patient.

However, in many situations the limits or thresholds for the triggering of the alarms are set close to the ranges of normal fluctuations of the values for the parameters being monitored, providing a safety net to prevent adverse events from being missed. As such, even when the parameter value only drops below the alarm limit for an instant due to a non-critical event, the device will trigger an alarm based on that sensed value. While setting the alarm limit in this manner is a safeguard against any significant issue or clinically relevant alarm being missed, as a result of the closeness of the threshold or alarm limit to the normal or acceptable ranges for this parameter, a large number of clinically irrelevant alarms are generated as well. Further, it is not possible to differentiate the clinically relevant alarms from the clinically irrelevant alarms based on the parameter value alone, such that each alarm event must be acted on in the same manner by the medical personnel monitoring the patient.

One result of the large number of the clinically irrelevant alarm events is the unnecessary expenditure of personnel, time and resources in attending to the clinically irrelevant alarm events. Another result is that certain highly important clinical events could inadvertently be overlooked or missed amidst the normally much larger number of clinically irrelevant alarm events. This is often referred to as alarm fatigue and results from the constant representation of the alarm events in a similar manner that can cause certain events to become “lost” in the flood of alarms and associated information represented on the display screen of the particular device. However, while current physiologic limit alarms have a very high false positive rate, setting the limits wider can reduce false positives but at the risk of missing critical events. Further, the standard alarm limits for many of these devices are often not determined based on the particular condition of the patient being monitored, which can increase the number of clinically irrelevant alarms.

Therefore, in order to address alarm fatigue and reduce the number of clinically irrelevant alarms that are generated, it is desirable to develop a monitoring device and associated alarm adjustment system that operates to involve the patient being monitored in determining the nature of the alarm.

### BRIEF DESCRIPTION OF THE INVENTION

In various embodiments of the invention, a monitoring device, system and method for monitoring a patient includes a display screen on which data concerning the patient being monitored by the device is illustrated. The incoming data sensed by the device is represented on the display screen to enable an individual viewing the display screen to determine the current physiological parameters of the patient.

The device and display screen can also illustrate various alarm conditions or events, as determined by the device from the incoming data signals received by the device from sensors attached to the patient being monitored. The alarm conditions or events are triggered by the comparison of parameter limits stored within the device to the incoming data signals from the sensors.

Once an alarm condition has been sensed, the monitoring system can utilize reactive inputs that can be determined/selected by the physician for use by the system. These reactive inputs are used by the system after the determination of an alarm event or condition as a check by the monitoring system that the alarm event is an actual clinically relevant and/or actionable alarm event, such as by soliciting a response from the patient directly about the current condition of the patient.

According to one exemplary embodiment of the invention, a medical monitoring device for providing information about a patient operably connected to the device includes an electronic storage medium in which values for alarm thresholds for a number of physiological parameters to be monitored are stored, a central processing unit operably connected to the electronic storage medium and configured to receive incoming data signals concerning the parameters of the patient and to compare the incoming data signals to the alarm limits for the parameters to determine an alarm condition, one or more sensors operably connected to the central processing unit and adapted to be connected to the patient to obtain and transmit the incoming data signals on the parameters to be monitored from the patient to the central processing unit and a display operably connected to the central processing unit, the display including a display screen and a user interface configured to enable reactive inputs to be stored in the electronic storage medium, wherein the central processing unit is configured to employ the reactive inputs to solicit patient assistance in response to a sensed alarm condition.

According to one exemplary embodiment of the invention, a medical monitoring device for providing information about a patient operably connected to the device and soliciting patient assistance in the confirmation of a sensed alarm condition includes an electronic storage medium in which values for alarm thresholds for a number of physiological parameters to be monitored and alarm criteria for the technical criteria for the operation of the device or the sensors are stored, a central processing unit operably connected to the electronic storage medium and configured to receive incoming data signals concerning the parameters of the patient and incoming data signals concerning technical criteria for the operation of the device or the sensors and to compare the incoming data signals to the alarm limits for the parameters to determine a sensed alarm condition, one or more sensors operably connected to the central processing unit and adapted to be connected to the patient to obtain and transmit the incoming data signals on the parameters to be monitored from the patient to the central processing unit and a display operably connected to the central processing unit, the display including a display screen and a user interface configured to enable reactive inputs to be stored in the electronic storage medium, wherein the central processing unit is configured to solicit a response from the patient based on the reactive input in response to the sensed alarm condition.

According to another exemplary embodiment of the invention, a monitoring device operable to provide information on data obtained from sensors operably connected between a patient and the device includes a central processing unit configured to receive incoming data signals from a sensor connected to the central processing unit concerning a physiological parameter of the patient and incoming data signals concerning technical criteria for the operation of the device or the sensors and to compare the incoming data signals to a predetermined alarm threshold for the physiological parameter or to an alarm criteria for the technical criteria for the operation of the device or the sensors to determine a sensed alarm condition and electronic storage media operably connected to the central processing unit and selectively operable to store reactive inputs for use by the monitoring device, wherein the central processing unit is configured to automatically employ the reactive inputs to solicit patient assistance in the confirmation of the sensed alarm condition.

According to still another exemplary embodiment of the invention, a method for utilizing patient assistance in the determination of a clinically relevant alarm condition using a monitoring device including the steps of providing a monitoring device including a central processing unit configured to receive incoming data signals from a sensor concerning a physiological parameter of a patient or incoming data signals concerning technical criteria for the operation of the device or the sensors and to compare the incoming data signals to a predetermined alarm criterion for the physiological parameters and operation of the device and sensors and to compare the incoming data signals to a predetermined alarm criterion for the physiological parameters and operational criteria of the device and sensors to determine an alarm condition, a user interface operably connected to the central processing unit and electronic storage media operably connected to the central processing unit and adapted to store reactive inputs for use in the determination of an alarm condition, providing a reactive input into the electronic storage media, determining the existence of a sensed alarm indication based on a comparison of the incoming data signals and the alarm criterion and soliciting a response from the patient based on the reactive inputs after determining the existence of the sensed alarm condition.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The drawings illustrate the best mode presently contemplated of carrying out the disclosure. In the drawings:

FIG. 1 is a schematic view a patient utilizing a monitoring system according to an exemplary embodiment of the invention.

FIG. 2 is a schematic diagram of an embodiment of a method of use of the monitoring system for monitoring a patient according to another exemplary embodiment of the invention.

### DETAILED DESCRIPTION

In the following detailed description, reference is made to the accompanying drawings that form a part hereof, and in which is shown by way of illustration specific embodiments, which may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken in a limiting sense.

Exemplary embodiments of the invention disclosed herein relate to a monitoring system and method for monitoring the vital signs of a patient using sensors disposed on the patient and operably connected to a monitor. The vital signs or parameters that are sensed by the sensors are transmitted to the monitor and compared with alarm thresholds stored within electronic storage media for the monitor. When an alarm condition is determined by a comparison of the sensed vital parameter reaching or exceeding the stored alarm threshold for that parameter, the system can employ one or more reactive inputs stored in the system immediately after an alarm event is detected, to solicit patient assistance in the determination of whether the alarm condition is clinically relevant and enable medical personnel to appropriately respond to sensed alarm event(s)/condition(s).

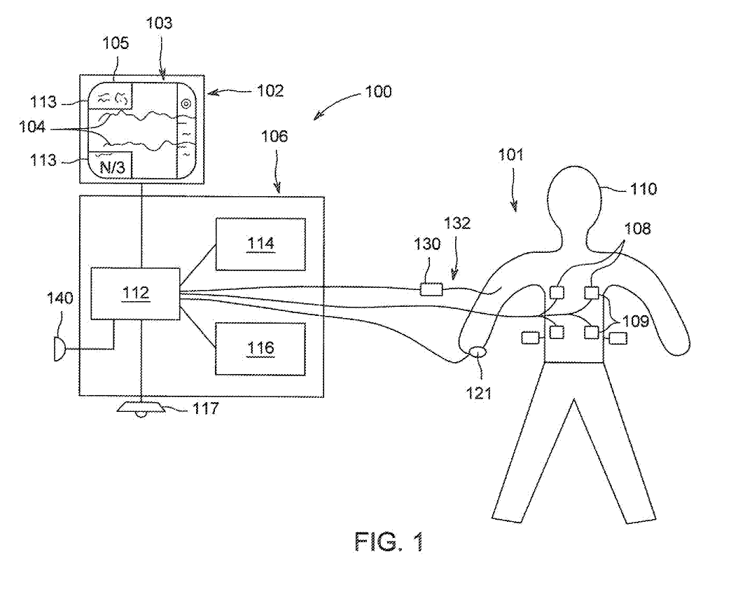

FIG. 1 illustrates an exemplary embodiment of the invention which includes a monitoring or diagnostic device and system 100, which can be any suitable type of monitoring device for monitoring various vital signs or parameters of an individual or other item 101 operably connected to the device 100. The device 100 includes a display 102 of any suitable type, such as a touch screen display, having a screen 103 thereon on which the monitoring data signals 104 regarding the object 101 connected to the device 100 can be displayed. When formed as a touch screen, the display 102 can additionally function as a user interface 105 for use in controlling the operation of the device 100, though the interface 105 can be formed as a separate component connected to the device 100, such as a keyboard (not shown) or mouse (not shown) if desired.

In the exemplary embodiment of FIG. 1, the device 100 takes the form of a medical monitoring device 106 that has one or more leads or sensors 108, such as impedance respiration/respiratory sensors 109, operably connected in any suitable manner between the medical monitoring device 106 and a patient 110 in order to monitor various vital statistics of the patient 110. In addition to the sensors 109, the device 100 can employ additional sensors 108 used to monitor other parameters or statistics of the patient 110, such as a pulse oximeter sensor 121 and/or an invasive pressure catheter 130 and invasive pressure transducer 132 to measure the blood flow and pressure of the patient 110 for comparison with the data obtained from the sensors 109, among others.

The medical monitoring device 106 includes a central processing unit (CPU) 112 operably connected to the sensors 108 in order to receive and process data from the sensors 108 on the various vital signs, statistics or parameters of specified bodily functions of the patient 110, which in the exemplary embodiment of FIG. 1 relates to respiratory functions, though other bodily functions or systems are also contemplated as being within the scope of the present invention. These parameter data can then be transmitted from the CPU 112 to the display 102 for presentation in a specified manner on the screen 103 of the display 102 for review by an individual monitoring the patient 110 via the display 102. Alternatively, the CPU 112 can be operably connected, such as by wired or wireless connections, to a network (not shown) that permits the use of multiple visual interfaces (not shown) including those such as a television, health monitor, iPhone or similar device, laptop, portable electronics, among others. This integration of the monitoring device 100 into a system with personal computing devices and portable electronics expands the communication capabilities between clinicians, as well as to facilitate patient observation from remote locations, e.g., central staff stations.

The device 100 also includes memory module 114, which can take the form of any suitable computer-readable electronic storage media, for example a RAM module, and an analytics engine 116, each of which are operably connected to the CPU 112 in order to assist in the monitoring function of the device 100 using the data signals 104 supplied to the CPU 112 via the sensors 108. The device 100 also includes an audio speaker 117 and microphone 140 for enabling the device 100 to provide and receive audible indications of various operating characteristics of the device 100.

The storage media 114 can include certain information regarding the predetermined normal or acceptable ranges for the operating parameters, vital statistics or physiological parameters for the patient 110 to which the device 100 is connected. These stored ranges can be utilized by the CPU 112 in conjunction with the incoming data signals 104 from the sensors 108 and the personal statistics of the patient 110 to determine the current vital statistics or physiological parameters of the patient 110 and whether those vital statistics or physiological parameters are outside of the predetermined ranges for those particular vital statistics or physiological parameters. The stored ranges for the incoming data signals 104 on the different physiological parameters and vital statistics received by the CPU 112 from the various sensors 108 can include ranges for minimum and maximum absolute values of the sensed parameters, minimum and maximum frequency ranges for the sensed physiological parameters, or any other suitable aspect of the incoming data signals 104 to be used in the determination of an alarm event or condition.

The storage media 114 can be accessed by a physician utilizing the interface 105 in order to provide or select one or more different reactive inputs that are to be utilized by the monitoring device 106 with regard to the alarm determination for the patient 110 connected to the monitoring device 106. The reactive inputs do not alter the alarm threshold(s) for any parameter being sensed by the monitoring device 106. Instead the monitoring device 106 employs the reactive inputs either just prior to or after an alarm condition/event has been triggered based upon data or other criteria sensed or determined by the device 106. This alarm condition can be based on the physiological signals received from the sensors 108 by the device 106 and compared against the alarm threshold(s) set within the monitoring device 106. In addition, the alarm condition can be based on other technical criterion, aspects or parameters of the operation of the device 106 and/or the sensors 108 that are determined by the device 106, such as when a sensor 108 is inadvertently disconnected from either the patient 110 or the device 106. The reactive inputs are employed to actively solicit a response or reaction from the patient 110 in response to the sensed or determined alarm condition, e.g., the physiological or technical alarm condition, in order to assess the severity or clinical relevancy of the sensed alarm condition. Based on the response received from the patient 110, the device 106 can then make a determination on the nature of the alarm event/condition as to whether it requires attention by medical personnel, such as by determining if the reaction of the patient 110 correct the alarm condition, by comparing the response received against a list of potential responses saved in storage media 114 or by employing rules provided to the analytics engine 116 for other decision making purposes, among other suitable determinations. Some examples of these reactive inputs include, but are not limited to:
i. Patient Direct Feedback/Response—when the alarm event is imminent or has been detected, the device 106 will prompt the patient 110 on the display 102 or via the speaker 117 to provide a direct response to the device 106, such as by touching the user interface 105 or another suitable direct response, such as by reconnecting a detached sensor 109,121, etc.;
ii. Patient Indirect Feedback/Response—the device 106 will prompt the patient 110 such as on the display 102 or via the speaker 117 to provide an indirect response to the device 106, such as by asking the patient 110 to move such that the movement sensed by the device 106 confirms patient 110 is breathing;
iii. Telepresence—the medical personnel can directly contact the patient 110 via the device 106, e.g., a video call using the display 102 or through the speaker 117, and inquire about the status or condition of the patient 110 to which the patient 110 can provide a response in a similar manner, such as through the microphone 140 or a communication hub (not shown) that is usable by the patient 110 to provide the response.

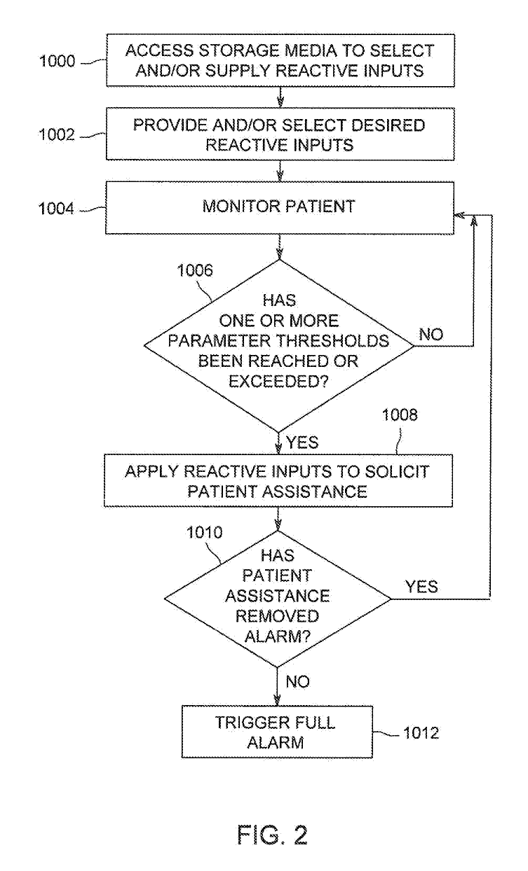

In an exemplary method associated with the system 100, device 106 and reactive inputs, as shown in FIG. 2 initially in block 1000 the physician or other medical personnel will access the storage media 114 in the device 106 using the interface 105. Depending upon the particular condition of the patient 110 to be monitored, in block 1002 the medical personnel will provide the desired reactive inputs to the device 106 for storage in the storage media 114 that correspond to the condition(s) and other monitoring/monitored parameters of the patient 110, such as those discussed previously. Alternatively, or in conjunction with the reactive inputs selected or provided by the medical personnel, the device 106 can have a standardized list of reactive inputs predetermined within the storage media 114 for use in monitoring a patient 110 which can optionally be modified by the physician as desired. Then in block 1004 the device 106 actively monitors the patient 110.

In performing the monitoring function in block 1004, the device 106 uses the parameter threshold(s) and other technical criterion/criteria for the operation of the device 106 stored in the storage media 114 to compare the incoming data from the sensors 108 on the vital signs/parameters of the patient 110 to be sensed and the technical information regarding the operation of the device 106 and/or the sensors 108. This data is then compared in decision block 1006 with the alarm threshold(s) and/or other technical criteria in order to continually determine if an alarm event or condition has been detected.

If no parameter alarm threshold or technical criteria is reached or exceeded, the system 100 returns to block 1004 to continue to monitor the vital parameters of the patient 110 and operation of the device 106 and sensors 108. However, if one of the sensed parameters has reached or exceeded the threshold for that parameter and/or if the technical criteria for the operation of the device 106 and/or sensors 108 has been violated in block 1008 the device 106 can proceed to utilize the reactive inputs stored in storage media 114. In accessing these reactive inputs in block 1008 the device 106, under the operational control of the CPU 112 and/or analytics engine 116, provides a prompt to the patient 110 in order to solicit a response from or obtain assistance from the patient in assessing the current condition of the patient 110 in relation to the sensed alarm event. The device 106 then interprets the response received from the patient 110 in decision block 1010 to determine if the response is sufficient to negate the alarm condition previously sensed, in which case the system 100 returns to block 1004 top continue monitoring the patient 110. Alternatively, if the response or assistance received from the patient or lack of response requires the alarm condition be confirmed to the appropriate medical personnel, the system 100 proceeds to trigger an alarm in block 1012.

With regard to the prompt provided to the patient 110 by the device 106 in response to a sensed alarm condition in block 1008, in certain exemplary embodiments these can be an automatic request by the device 106 directly (without any intervention by a physician or other medical personnel), or a live request by a physician/medical personnel to the patient 110 to take certain corrective action, if appropriate. The corrective action requested can be to check the attachment of the sensor 108 to the patient 110 that is causing the alarm condition, or to request a response to a question provided by a physician/medical personnel through an active telecommunication link in the device 106, such as through the speaker 117 and an associated microphone 140. This response from the patient 110 can indicate to the physician/medical personnel what the patient 110 is currently feeling to provide an accurate indication of the current condition of the patient 110 to the physician/medical personnel outside of the sensed alarm condition.

In addition to the previously described exemplary embodiment, the system 100 including the reactive inputs stored and/or provided to system 100 can be applied to systems 100 that employ monitoring devices worn by the patient (not shown) and different systems 100 having sensors 108 and hubs (not shown) in the control of the patient 110.

The written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A method for utilizing patient assistance in the determination of a clinically relevant alarm condition using a monitoring device, the method comprising the steps of:
a. providing a monitoring device including a central processing unit configured to receive incoming data signals from a sensor concerning a physiological parameter of a patient or incoming data signals concerning technical criteria for the operation of the device or the sensors and to compare the incoming data signals to a predetermined alarm criterion for the physiological parameters and operational criteria of the device and sensors to determine an alarm condition, a user interface operably connected to the central processing unit and electronic storage media operably connected to the central processing unit and adapted to store reactive inputs for use in the determination of an alarm condition;
b. providing a reactive input into the electronic storage media;
c. determining the existence of a sensed alarm indication based on a comparison of the incoming data signals and the alarm criterion; and
d. soliciting a response from the patient based on the reactive inputs during the sensed alarm condition after determining the existence of the sensed alarm condition.

2. The method of claim 1 wherein the step of soliciting a response comprises providing a prompt to the patient to provide an indication of the current condition of the patient.

3. The method of claim 2 wherein the step of providing the prompt comprises requesting the patient to take corrective action regarding the sensed alarm condition.

4. The method of claim 3 wherein the step of requesting the patient to take corrective action comprises requesting the patient to re-attach a disengaged sensor.

5. The method of claim 2 wherein the step of providing the prompt to the patient comprises providing an automatic request to the patient.

6. The method of claim 2 wherein the step of providing the prompt to the patient comprises providing a live request to the patient.

7. The method of claim 6 wherein the step of providing a live request to the patient comprises:
opening a telecommunication link between the patient and the physician/medical personnel via the device

8. The method of claim 1 wherein the step of soliciting a response from the patient comprises actively querying the patient.

9. The method of claim 8 wherein the step of actively querying the patient comprises prompting the patient to provide an audible response.

10. The method of claim 1 wherein the step of soliciting a response from the patient comprises indirectly querying the patient.

11. A monitoring device operable to provide information on data obtained from sensors operably connected between a patient and the device, the device comprising:
a) a central processing unit configured to receive incoming data signals from a sensor connected to the central processing unit concerning a physiological parameter of the patient and incoming data signals concerning technical criteria for the operation of the device or the sensors and to compare the incoming data signals to a predetermined alarm threshold for the physiological parameter or to an alarm criteria for the technical criteria for the operation of the device or the sensors to determine a sensed alarm condition; and
b) electronic storage media operably connected to the central processing unit and selectively operable to store reactive inputs for use by the monitoring device, wherein the central processing unit is configured to automatically employ the reactive inputs during the sensed alarm condition to solicit patient assistance in the confirmation of the sensed alarm condition.

12. The monitoring device of claim 11 wherein the central processing unit is configured to automatically solicit a response from the patient upon the determination of an alarm condition by the central processing unit.

13. The monitoring device of claim 12 wherein the central processing unit is configured to automatically solicit a direct response from the patient.

14. The monitoring device of claim 13 wherein the central processing unit is configured to automatically solicit corrective action from the patient.

15. The monitoring device of claim 13 wherein the central processing unit is configured to automatically solicit a verbal response from the patient.

16. A medical monitoring device for providing information about a patient operably connected to the device and soliciting patient assistance in the confirmation of a sensed alarm condition, the device comprising:
a) an electronic storage medium in which values for alarm thresholds for a number of physiological parameters to be monitored and alarm criteria for the technical criteria for the operation of the device or the sensors are stored;
b) a central processing unit operably connected to the electronic storage medium and configured to receive incoming data signals concerning the parameters of the patient and incoming data signals concerning technical criteria for the operation of the device or the sensors and to compare the incoming data signals to the alarm limits for the parameters to determine a sensed alarm condition;
c) one or more sensors operably connected to the central processing unit and adapted to be connected to the patient to obtain and transmit the incoming data signals on the parameters to be monitored from the patient to the central processing unit; and
d) a display operably connected to the central processing unit, the display including a display screen and a user interface configured to enable reactive inputs to be stored in the electronic storage medium, wherein the central processing unit is configured to solicit a response from the patient based on the reactive input in response to and during the sensed alarm condition.

17. The method of claim 1 further comprising the step of remotely silencing an alarm after soliciting the response from the patient.
